(19) United States
(12) Patent Application Publication
(10) Pub. No.: US 2026/0183247 A1
(43) Pub. Date: Jul. 2, 2026

(54) GENE DELIVERY NANOPARTICLE, AND PREPARATION AND APPLICATION THEREOF

(71) Applicant: Institute of Radiation Medicine, Chinese Academy of Medical Sciences, Tianjin (CN)

(72) Inventors: Huijuan SONG, Tianjin (CN); Qiang LIU, Tianjin (CN); Ningning HE, Tianjin (CN); Feifei MA, Tianjin (CN); Tuo LI, Tianjin (CN); Siyu XIE, Tianjin (CN); Yi REN, Tianjin (CN)

(21) Appl. No.: 19/416,514

(22) Filed: Dec. 11, 2025

(30) Foreign Application Priority Data
Dec. 26, 2024 (CN) 202411936960.9

(51) Int. Cl. A61K 9/51; A61K 47/69

(52) U.S. Cl. CPC A61K 9/5192; A61K 9/5146; A61K 47/6935

(57) ABSTRACT

The present disclosure provides a gene delivery nanoparticle, and its preparation and application, pertaining to the technical field of biomedicine. In the preparation method, 2-((tert-butoxycarbonyl)amino)ethyl methacrylate as monomer, mPEG₅ₖ-RAFT as macromolecular chain transfer agent and azobisisobutyronitrile as initiator are subjected to reversible addition-fragmentation chain transfer polymerization in dimethylformamide, dialysis and freeze-drying to obtain an intermediate; the intermediate is dissolved in dichloromethane, added with trifluoroacetic acid and reacted to yield polyethylene glycol-poly(aminoethyl methacrylate); sparfloxacin is dissolved in a hydrochloric acid solution, and added with 1-(3-dimethylaminopropyl)-3-ethylcarbodiimide hydrochloride, N-hydroxysuccinimide and a solution of polyethylene glycol-poly(aminoethyl methacrylate) in dimethyl sulfoxide to produce a mixture; the mixture is subjected to dialysis and freeze-drying to obtain the final product polyethylene glycol-poly(sparfloxacin), which is placed in an aqueous solution to allow self-assembly, thereby forming the desired nanoparticle.

### CROSS-REFERENCE TO RELATED APPLICATIONS

The present application claims priority to Chinese Patent Application No. 202411936960.9, filed on Dec. 26, 2024, the entire disclosure of which is incorporated herein by reference.

### TECHNICAL FIELD

The present disclosure relates to the field of biomedicine technology, and in particular to a gene delivery nanoparticle for dendritic cells.

### BACKGROUND

Immunotherapy has emerged as a highly effective therapy for cancer treatment following surgery, radiotherapy, chemotherapy and targeted therapy. It has demonstrated breakthrough efficacy in the treatment of recurrent and refractory tumors, becoming an important part of the comprehensive cancer management. Dendritic cells (DCs) serve as crucial antigen-presenting cells within the immune system. They are capable of capturing, processing, and presenting antigens to T cells, thereby initiating immune responses and enhancing anti-tumor immunity.

DCs can capture antigenic substances released by tumor cells, including proteins, peptides, or other molecules present on the tumor cell surface, through surface receptors, and then process and degrade the captured antigens into small peptide fragments, which are combined with major histocompatibility complex (MHC) molecules to form antigen-MHC complexes. These processed antigen-MHC complexes are carried by DCs as they migrate to lymphoid organs, such as lymph nodes. Within lymphoid tissues, DCs interact with T cells, and provide the signals necessary for T cell activation through the interaction of co-stimulatory molecules on DCs with their corresponding receptors on T cells. Simultaneously, the antigen-MHC complexes are presented to T cells, enabling the antigen-specific recognition and binding. Activated effector T cells possess the ability to kill tumor cells or regulate immune responses. Some activated T cells differentiate into immunological memory T cells, which can persist for a long term and can rapidly initiate the immune responses upon re-encountering the same antigen, thereby conferring the sustained anti-tumor immunity. Through this process, DCs can efficiently capture, process, and present tumor antigens, activating the T cell-mediated immune responses and directing the anti-tumor immunity. Furthermore, DCs can further enhance the anti-tumor immune responses by secreting cytokines and chemokines to modulate the function of immune cells.

To enhance the anti-tumor immune response mediated by DCs, various strategies have been investigated, such as optimizing DC culture and activation conditions, loading DCs with tumor-specific antigens or antigenic peptides, and combining DC-based therapy with other immunotherapeutic approaches. These efforts aim to improve the immunostimulatory capacity of DCs and facilitate the development of more effective anti-tumor immunotherapy.

Gene delivery-mediated activation of DCs represents an immunotherapeutic strategy that involves genetically modifying DCs to enhance their antigen-presenting ability and immunostimulatory activity, thereby triggering a potent anti-tumor immune response. Genes—which may be in the form of DNA, RNA, or other biological molecules—can be combined with nanoparticles through physical adsorption, chemical binding, or electrostatic interactions. Through the gene delivery techniques, specific genes can be introduced into DCs to enable the expression of immune-stimulatory molecules, co-stimulatory molecules or tumor antigens, ultimately enhancing the ability to activate the immune response.

For example, genes encoding immunostimulatory molecules (such as cytokines or co-stimulatory molecules) can be introduced into DCs, enabling the expression of these molecules on the cell surface. This enhances the DC-T cell interactions and promotes the activation and proliferation of T cells. Alternatively, loading DCs with genes encoding tumor antigens enables the expression of tumor-associated antigens, and then these engineered DCs can be reinfused into patients to induce a specific anti-tumor immune response. Furthermore, gene delivery techniques can be employed to modulate the DC function—for instance, by enhancing their antigen uptake capacity or promoting their maturation and migration—thereby improving the immunostimulatory efficacy of DCs. Researches on gene delivery-based activation of DCs hold significant promise for advancing the cancer immunotherapy.

However, this technology still faces several challenges, such as low gene transfection efficiency, unstable gene expression, and difficulties in maintaining the survival and function of DCs. Currently, an ideal gene delivery carrier with low toxicity and high efficiency is still absent in the clinical practice.

### SUMMARY

An object of the present disclosure is to provide a gene delivery nanoparticle, and a preparation and application thereof to overcome the above defects in the prior art.

In order to address the above problems, the present disclosure provides a method for preparing a gene delivery nanoparticle, comprising:
subjecting 2-((tert-butoxycarbonyl)amino)ethyl methacrylate as a monomer, methoxy poly(ethyleneglycol)₅ₖ-reversible addition-fragmentation chain transfer agent (mPEG₅ₖ-RAFT) as a macromolecular chain transfer agent and azobisisobutyronitrile as an initiator to a reversible addition-fragmentation chain-transfer polymerization reaction in dimethylformamide, followed by dialysis and freeze-drying to obtain an intermediate;
dissolving the intermediate in dichloromethane, followed by addition of trifluoroacetic acid and reaction to obtain polyethylene glycol-poly(aminoethyl methacrylate);
dissolving sparfloxacin in a hydrochloric acid solution, followed by addition of 1-(3-dimethylaminopropyl)-3-ethylcarbodiimide hydrochloride and N-hydroxysuccinimide to produce a first solution; dissolving the polyethylene glycol-poly(aminoethyl methacrylate) in dimethyl sulfoxide to produce a second solution; adding the second solution to the first solution to produce a mixture; and subjecting the mixture to dialysis and freeze-drying to obtain a final product polyethylene glycol-poly(sparfloxacin); and
placing the polyethylene glycol-poly(sparfloxacin) in an aqueous solution for self-assembly to form the gene delivery nanoparticle.

In an embodiment, a concentration of the polyethylene glycol-poly(sparfloxacin) is 1-40 mg/mL.

In an embodiment, the polyethylene glycol-poly(sparfloxacin) is capable of self-assembling into an empty nanoparticle or a bis[2,4,5-trichloro-6-(pentyloxycarbonyl)phenyl] oxalate (CPPO) nanoparticle in the presence of CPPO.

In an embodiment, a concentration of CPPO is 0.5-20 mg/mL.

The present disclosure further provides a novel gene delivery nanoparticle, wherein the gene delivery nanoparticle is prepared by the above preparation method, and is a nanoparticle self-assembled from polyethylene glycol-poly(sparfloxacin); and the polyethylene glycol-poly(sparfloxacin) is structurally represented by

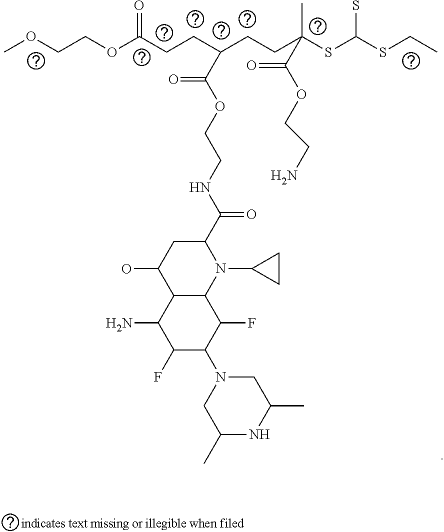 [SMILES: CCSC(S)SC(C)(CCC(CCC(=O)OCCOC)C(=O)OCCNC(=O)C1CC(O)C2C(N)C(F)C(N3CC(C)NC(C)C3)C(F)C2N1C1CC1)C(=O)OCCN]

In an embodiment, the nanoparticle has a particle size of 50-250 nm.

In an embodiment, the nanoparticle is capable of binding to immunoadjuvant cytosine-phosphorothioate-guanine oligodeoxynucleotides (CpG ODN), programmed death ligand-1 small interfering RNA (PD-L1 siRNA) and ovalbumin (OVA) mRNA via electrostatic adsorption.

In an embodiment, a weight ratio of the polyethylene glycol-poly(sparfloxacin) to the immunoadjuvant CpG ODN for binding is no less than 3:1, a weight ratio of the polyethylene glycol-poly(sparfloxacin) to the PD-L1 siRNA for binding is no less than 1:1, and a weight ratio of the polyethylene glycol-poly(sparfloxacin) to the OVA mRNA for binding is no less than 3:1.

In an embodiment, the gene delivery nanoparticle is capable of serving as a carrier to transfect a plasmid into dendritic cells.

The present disclosure further provides an application of a novel gene delivery nanoparticle in the treatment of a tumor, wherein the nanoparticle is the gene delivery nanoparticle described in the above technical solutions.

The present disclosure provides a gene delivery nanoparticle, and its preparation and application. The preparation method of the novel dendritic cell (DC)-activating gene delivery nanoparticle is simple and easy to operate. It is formed via self-assembly driven by interactions between the hydrophilic and hydrophobic segments of the copolymer, without involving complex chemical reactions or toxic organic reagents, which is beneficial for production and clinical application. The present disclosure proposes a new strategy to enhance the gene delivery efficiency in DCs. Through the RAFT polymerization, a long-circulating sparfloxacin prodrug in vivo is prepared. By employing self-assembly technology, CPPO is efficiently loaded, resulting in CPPO-loaded nanoparticles. An appropriate concentration of polyethylene glycol-poly(sparfloxacin) can increase the level of reactive oxygen species (ROS) in DCs. The CPPO within the nanoparticles can cleave to release energy, activating the aggregated sparfloxacin to generate higher levels of ROS. This promotes the endosomal escape of the delivered CpG, siPD-L1, and OVA-mRNA genes, thereby effectively activating and facilitating the maturation of DCs, and inducing a systemic immune response. The present disclosure provides technical support for accelerating the clinical application of nanomedicine-mediated immunotherapy.

### BRIEF DESCRIPTION OF THE DRAWINGS

To provide a clearer illustration of the technical solutions in the embodiments of the present disclosure or the prior art, the accompanying drawings required for describing the embodiments or the prior art will be briefly introduced below. Obviously, the drawings in the following description represent only some embodiments of the present disclosure. For those of ordinary skill in the art, other drawings may be derived from the provided drawings without paying creative effort.

FIG. 1 schematically shows a synthesis route of a polyethylene glycol-poly(sparfloxacin) (PESPA) copolymer provided in Example 1.

FIG. 2 is a nuclear magnetic resonance (NMR) spectrum of the PESPA copolymer provided in Example 1.

FIG. 3 shows ultraviolet (UV) spectra of sparfloxacin (SPA), PESPA, bis[2,4,5-trichloro-6-(pentyloxycarbonyl)phenyl] oxalate (CPPO), and PESPA@CPPO nanoparticles provided in Example 2.

FIG. 4 shows zeta potentials of PESPA and PESPA@CPPO nanoparticles in Example 2.

FIG. 5 displays the particle size distribution of PESPA and PESPA@CPPO nanoparticles in Example 2, as determined by dynamic light scattering (DLS), along with transmission electron microscopy (TEM) images obtained using an electron beam source.

FIG. 6 displays the particle sizes of PESPA and PESPA@CPPO nanoparticles in Example 2, as measured by dynamic light scattering (DLS) in PBS and in PBS containing 10% fetal bovine serum (FBS).

FIGS. 7a-7b illustrate the effect of PESPA and PESPA@CPPO nanoparticles in Example 3 on reactive oxygen species (ROS) levels in DC2.4 cells.

FIG. 8 shows the DNA gel electrophoresis results of CpG bound to PESPA and PESPA@CPPO nanoparticles at different mass ratios, as investigated in Example 4.

FIGS. 9a-9b demonstrate the effect of PESPA@CpG and PESPA@CPPO@CpG nanoparticles from Example 5 on promoting the maturation of BMDC cells.

FIG. 10 presents the DNA gel electrophoresis results of OVA-mRNA bound to PESPA and PESPA@CPPO nanoparticles at different mass ratios, as investigated in Example 6.

FIGS. 11a-11c demonstrates the ability of PESPA@OVA mRNA and PESPA@CPPO@OVA mRNA nanoparticles from Example 7 to promote BMDC cell maturation and antigen cross-presentation.

FIG. 12 shows the DNA gel electrophoresis results of siPD-L1 bound to PESPA@CPPO nanoparticles at different mass ratios, as investigated in Example 8.

FIG. 13 shows fluorescence images reflecting transfection of plasmid H128 pLenti-EF1a-EGFP-3FLAG-PGK-Puro mediated by PESPA and PESPA@CPPO nanoparticles from Example 9.

### DETAILED DESCRIPTION OF THE EMBODIMENTS

To make the aforementioned objectives, features, and advantages of the present disclosure more clearly and understandable, the specific embodiments of the disclosure will be described in detail below with reference to the accompanying drawings. It should be understood that the specific embodiments described herein are merely intended to illustrate the disclosure, and should not be construed as limiting the scope of the disclosure.

### Example 1 Synthesis of Copolymer PESPA

The copolymer polyethylene glycol-poly(sparfloxacin), designated as PESPA, was prepared as follows.

First, a macromolecular chain transfer agent mPEG₅ₖ-RAFT (0.26735 g, 0.05 mmol) was dissolved in 4 mL of dimethylformamide (DMF). Then, 2-((tert-butoxycarbonyl)amino)ethyl methacrylate (1.14565 g, 5 mmol) and an initiator azobisisobutyronitrile (AIBN) (1.3421 mg, 0.01 mmol) were added. The reaction mixture was subjected to three cycles of vacuumization and argon purging, and then underwent a reversible addition-fragmentation chain transfer (RAFT) polymerization at 70° C. for 24 hours. The resultant reaction product was subjected to dialysis and freeze-drying to obtain an intermediate.

The intermediate was dissolved in 5 mL of dichloromethane (DCM), added with 5 mL of trifluoroacetic acid (TFA) and stirred at room temperature for 6 hours to yield the polymer polyethylene glycol-poly(aminoethyl methacrylate), designated as PEPA.

Sparfloxacin (SPA) (2.3544 g, 6 mmol) was dissolved in 10 mL of hydrochloric acid solution, to which 1-(3-dimethylaminopropyl)-3-ethylcarbodiimide hydrochloride (EDC) (1.7253 g, 9 mmol) and N-hydroxysuccinimide (NHS) (2.07144 g, 18 mmol) were added. The PEPA polymer was dissolved in 5 mL of dimethyl sulfoxide (DMSO) and then added to the aforementioned aqueous solution. The reaction mixture was reacted at room temperature for 5 days, followed by dialysis and freeze-drying to give the final product PESPA.

The resulting polymer was chemically characterized by NMR and mass spectrometry.

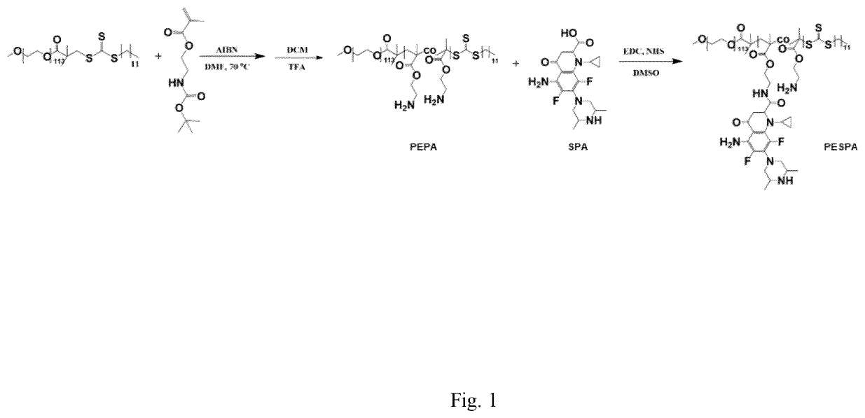

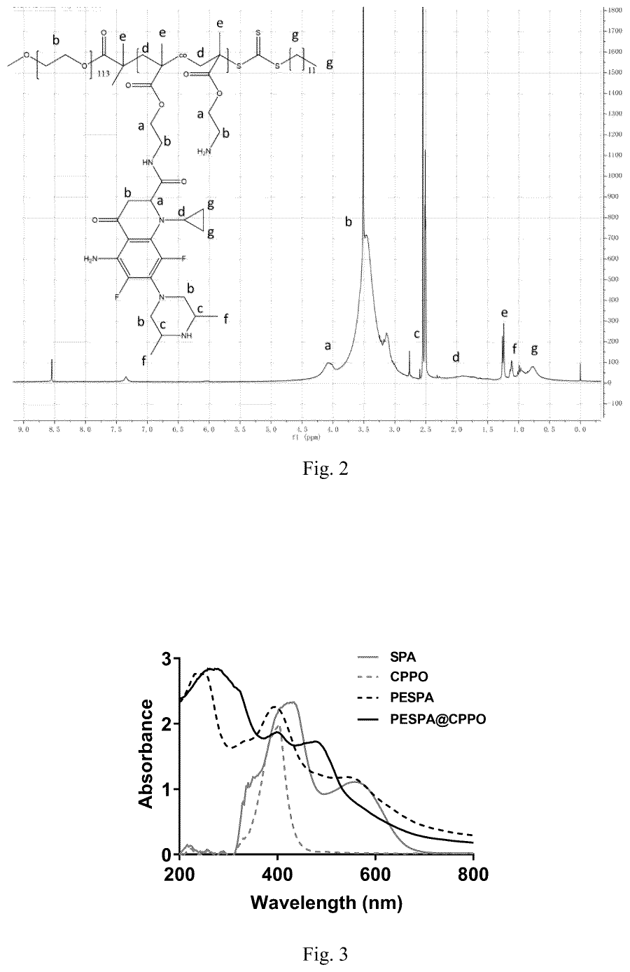

FIG. 1 illustrated the synthesis scheme of the PESPA copolymer in this example; and FIG. 2 presented the NMR spectrum of the PESPA copolymer. The characteristic resonance peaks of both PEG and SPA can be clearly observed in FIG. 2, confirming the successful synthesis of the PESPA copolymer and clearly defining the structure and composition.

### Example 2 Preparation of PESPA NPs and PESPA@CPPO NPs

The unloaded nanoparticles were designated as PESPA NPs, and those loaded with CPPO were designated as PESPA@CPPO NPs.

20 mg of the PESPA polymer was dissolved in 1 mL of 2,2,2-trifluoroethanol (TFE). For the preparation of the loaded particles, 20 mg of the PESPA polymer and 10 mg of CPPO were co-dissolved in 1 mL of TFE. This solution was added dropwise at a rate of 100 μL/min to 10 mL of Milli-Q water under stirring. The resulting solution was stirred continuously for 48-72 hours to allow for complete evaporation of TFE, and then centrifuged at 3,000 rpm for 5 minutes to obtain PESPA NPs and PESPA@CPPO NPs.

The nanoparticles were diluted with Milli-Q water to a concentration of 1 mg/mL, from which a 10-20 μL sample was collected dropped onto a 230-mesh copper grid coated with a carbon support film and dried at constant temperature. The grid was stained with 5 μL of a 1-2% uranyl acetate solution for 5 minutes (with the excess staining solution removed by filter paper), dried at room temperature and observed using a Transmission Electron Microscope (TEM, FEI Tecnai G220S-TWIN, USA) at an accelerating voltage of 200 kV for the sample morphology.

The nanoparticles were diluted with Milli-Q water to a concentration of 1 mg/mL. The particle size and zeta potential were measured using a Dynamic Light Scattering instrument (DLS, Malvern Zetasizer Nano ZS, UK) at 25° C. with a detection angle of 90° and a laser wavelength of 633 nm. Prior to each measurement, the sample was allowed to equilibrate for 2 minutes. Each sample was measured three times, and the results were averaged.

The nanoparticles were diluted with Milli-Q water to a concentration of 0.1 mg/mL. The absorption peak was measured using a UV-Vis spectrophotometer at 25° C., with a scanning wavelength range from 200 to 800 nm and a detection angle of 90°. Each sample was measured three times, and the results were averaged.

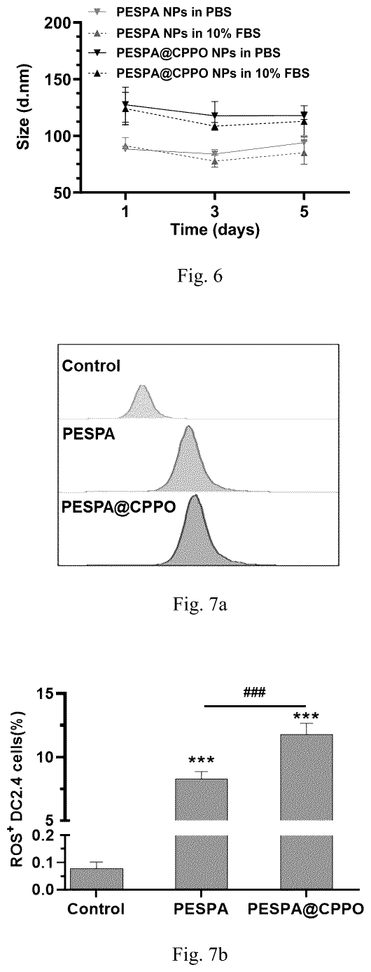

The corresponding characterization spectra were shown in FIGS. 3-6. Specifically, FIG. 3 presented the UV spectra of both PESPA NPs and PESPA@CPPO NPs in this example, from which it can be clearly observed that the absorption peak of PESPA@CPPO NPs overlaps with that of free CPPO at around 400 nm, confirming the successful loading of CPPO into the nanoparticles.

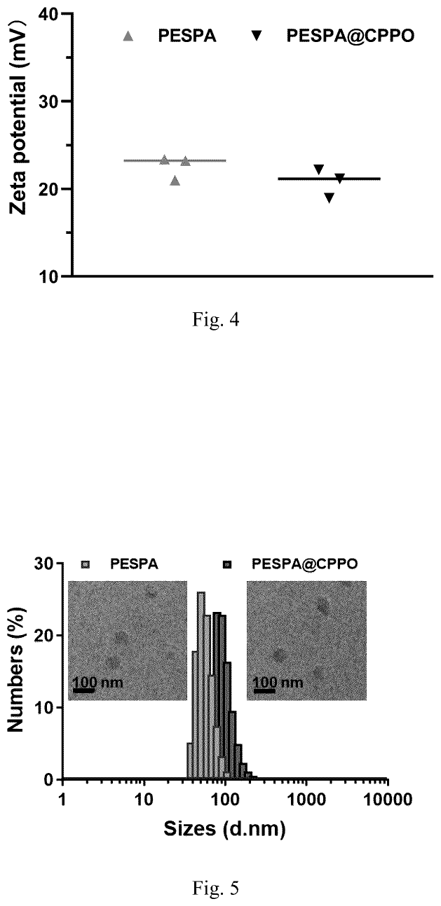

FIG. 4 displayed the zeta potential profiles of PESPA NPs and PESPA@CPPO NPs in this example. As shown in FIG. 4, both nanoparticles were positively charged, with measured values of approximately +22.52 mV and +20.74 mV, respectively.

FIG. 5 presented the DLS size distribution of PESPA NPs and PESPA@CPPO NPs in this example, from which it can be observed that the average particle sizes were approximately 60 nm and 80 nm for PESPA NPs and PESPA@CPPO NPs, respectively. As evidenced by the TEM images in FIG. 5, both nanoparticles exhibited a well-defined spherical morphology. Furthermore, the particle sizes observed via TEM were consistent with the results determined by DLS.

### Example 3 Effect of PESPA NPs and PESPA@CPPO NPs on the Reactive Oxygen Species (ROS) Level in DC2.4 Cells

To investigate the effects of PESPA NPs and PESPA@CPPO NPs on the ROS level in DC2.4 cells, the cells were seeded in a 12-well plate at a density of 8×10⁴ cells per well and divided into three groups: control, PESPA NPs, and PESPA@CPPO NPs. The nanoparticles (PESPA NPs and PESPA@CPPO NPs) were administered at a concentration of 20 μg/mL. After 24 hours of treatment, the ROS level in DC2.4 cells was measured using the fluorescent probe 2′,7′-dichlorodihydrofluorescein diacetate (DCFH-DA).

FIGS. 7a-7b demonstrated the flow cytometry results of ROS levels in DC2.4 cells. Compared with the control group, both PESPA NPs and PESPA@CPPO NPs treatment groups showed a significant increase in the proportion of ROS-positive cells, indicating that both types of nanoparticles can effectively enhance the ROS levels in DC2.4 cells. Furthermore, at the same concentration, the PESPA@CPPO NPs induced a higher ROS level compared to the PESPA NPs.

### Example 4 Measurement of Binding Efficiency Between PESPA NPs/PESPA@CPPO NPs and CpG

To determine the binding efficiency of CpG with PESPA NPs and PESPA@CPPO NPs, CpG (1 mg/mL) was mixed with PESPA NPs (1 mg/mL) or PESPA@CPPO NPs (1 mg/mL) at different concentration ratios. After co-incubation at room temperature for 1 hour, the binding efficiency was assessed by 1% agarose gel electrophoresis under a constant voltage of 110 V for 20 minutes.

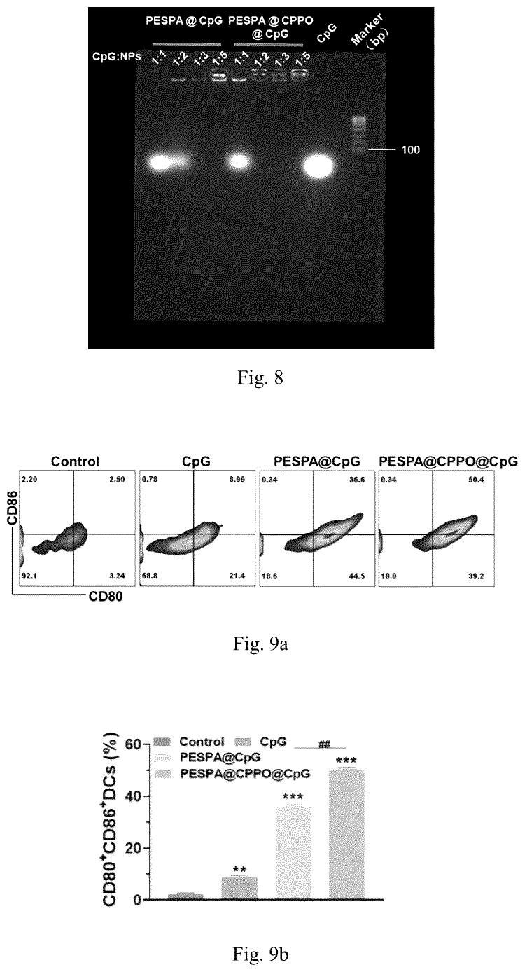

FIG. 8 presented the UV imaging results of the gel electrophoresis. Regardless of whether the nanoparticles were loaded with CPPO or not, no fluorescence was observed at the mixing ratios from 1:3 to 1:5. In contrast, a strong fluorescence signal was detected in the CpG positive control group. These results indicated that both PESPA and PESPA@CPPO nanoparticles successfully bound with CpG at all tested concentration ratios starting from 1:3.

### Example 5 Evaluation of the Maturation-Promoting Ability of PESPA@CPPO@CpG NPs on Bone Marrow-Derived Dendritic Cells (BMDCs)

Bone marrow-derived stem cells were isolated from C57 mice and cultured for 5 days in RPMI-1640 medium supplemented with GM-CSF, IL-4, and inactivated FBS to develop into bone marrow-derived dendritic cells (BMDCs). The BMDCs were then seeded in a 12-well plate at a density of 2×10⁵ cells per well and divided into four experimental groups: Control, CpG, PESPA@CpG, and PESPA@CPPO@CpG. The CpG group was treated with 2 μg/mL of CpG, while the nanoparticle-treated groups (PESPA@CpG and PESPA@CPPO@CpG) received nanoparticles at a concentration of 10 μg/mL. After 24 hours of treatment, the cells were incubated for 1 hour with CD11c, CD80, and CD86 antibodies, followed by analysis using flow cytometry.

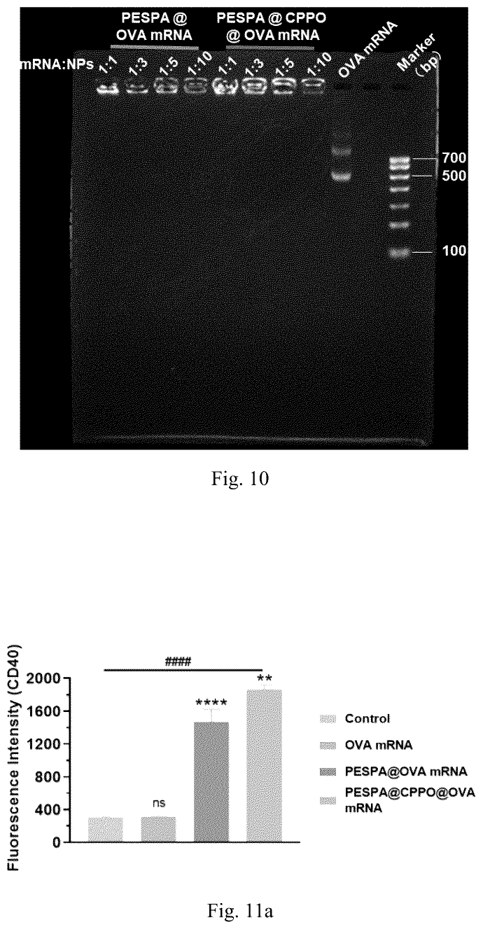

FIGS. 9a-9b presented the evaluation of the maturation-promoting effect of PESPA@CPPO@CpG NPs on BMDCs in this example. The results in FIG. 9 demonstrated that both PESPA@CpG and PESPA@CPPO@CpG NPs significantly increased the proportion of CD80⁺CD86⁺ BMDCs, indicating that these two types of nanoparticles can effectively promote the dendritic cell maturation.

### Example 6 Determination of Binding Efficiency Between PESPA NPs/PESPA@CPPO NPs and OVA mRNA

To assess the binding efficiency of OVA mRNA to PESPA NPs and PESPA@CPPO NPs, OVA mRNA (1 mg/mL) was mixed with the nanoparticles (1 mg/mL) at varying concentration ratios. After co-incubation at room temperature for 1 hour, the binding efficiency was evaluated by 1% agarose gel electrophoresis at a constant voltage of 110 V for 20 minutes.

FIG. 10 displayed the UV imaging results of the gel electrophoresis assay. Regardless of whether the nanoparticles were loaded with CPPO or not, no fluorescent bands were observed at mixing ratios ranging from 1:1 to 1:10. In contrast, the OVA mRNA positive control group exhibited a distinct fluorescent signal. These results confirmed the successful formation of complexes between OVA mRNA and both PESPA and PESPA@CPPO NPs across all tested concentration ratios (starting from 1:1).

### Example 7 Evaluation of Ability of PESPA@CPPO@OVA mRNA Nanoparticles to Promote Antigen Cross-Presentation of BMDCs

Bone marrow-derived stem cells were isolated from C57BL/6 mice and differentiated into BMDCs by culturing for 5 days in RPMI-1640 medium supplemented with GM-CSF, IL-4 and inactivated fetal bovine serum (FBS). The BMDCs were then seeded into a 12-well plate at a density of 2×10⁵ cells per well and divided into four treatment groups: Control, OVA mRNA, PESPA@OVA mRNA and PESPA@CPPO@OVA mRNA. The OVA mRNA group was treated with 2 μg/mL of OVA mRNA, while the nanoparticle groups (PESPA@OVA mRNA and PESPA@CPPO@OVA mRNA) were respectively treated with 10 μg/mL of the corresponding nanoparticle. After 24 hours of treatment, cells were incubated for 1 hour with CD40, CD86 and SIINFEKL-H-2Kb antibodies, followed by analysis using flow cytometry.

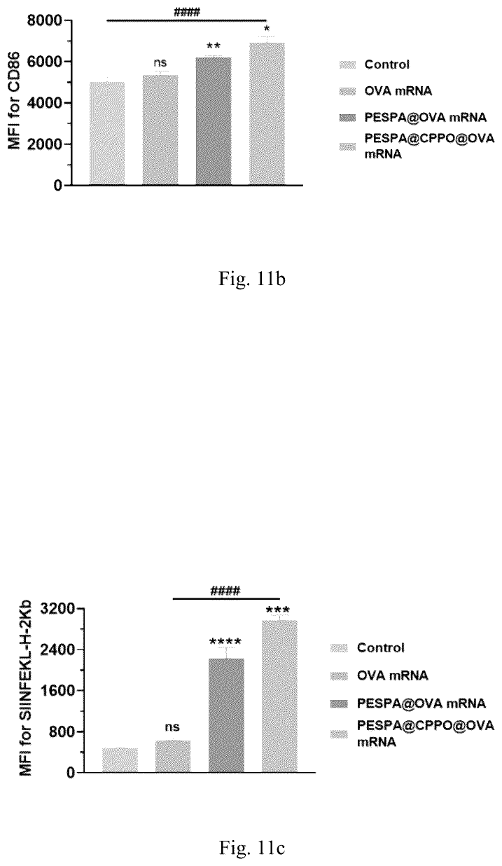

FIGS. 11a-11c showed the evaluation of the ability of the PESPA@CPPO@OVA mRNA NPs in Example 5 to promote the antigen cross-presentation of BMDCs, from which it can be demonstrated that both PESPA@OVA mRNA and PESPA@CPPO@OVA mRNA NPs significantly increased the fluorescence intensity of CD40 and CD86 in BMDCs, indicating that these two types of nanoparticles can effectively promote the dendritic cell maturation. Furthermore, the high expression level of SIINFEKL-H-2Kb in both nanoparticle-treated groups demonstrated that both nanoparticles can effectively promote the OVA mRNA antigen presentation by DCs.

### Example 8 Determination of the Binding Efficiency Between PESPA@CPPO NPs and Small Interfering RNA Targeting Programmed Cell Death Ligand 1 (siPD-L1)

To determine the binding efficiency between PESPA@CPPO NPs and siPD-L1, siPD-L1 (1 mg/mL) was mixed with PESPA@CPPONPs (1 mg/mL) at varying concentration ratios. After co-incubation at room temperature for 1 hour, the binding efficiency was assessed by 1% agarose gel electrophoresis at a constant voltage of 110 V for 20 minutes.

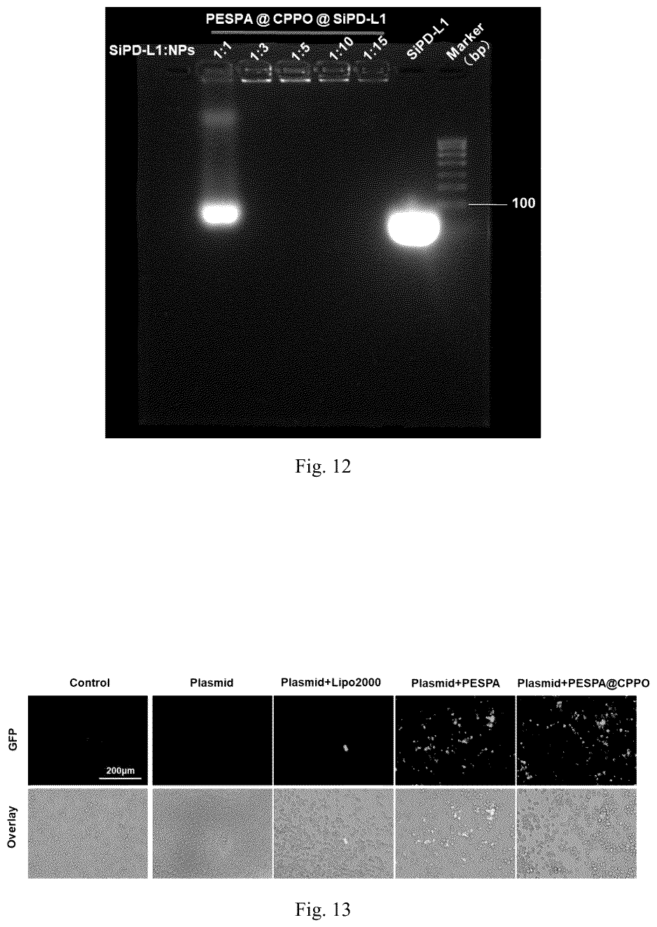

FIG. 12 showed the UV imaging results of the gel electrophoresis assay. No fluorescent bands were observed at mixing ratios ranging from 1:3 to 1:15, whereas the siPD-L1 positive control group exhibited a distinct fluorescent signal. These results confirmed the successful binding between siPD-L1 and PESPA@CPPO NPs across all tested concentration ratios (starting from 1:3).

### Example 9 Determination of Plasmid Transfection Efficiency of PESPA NPs and PESPA@CPPO NPs in DC2.4 Cells

To evaluate the plasmid transfection efficiency of PESPA NPs and PESPA@CPPO NPs in DC2.4 cells, the cells were seeded in a 12-well plate at a density of 8×10⁴ cells per well and divided into the following groups: Control, Plasmid, lipo2000+Plasmid, PESPA@Plasmid, and PESPA@CPPO@Plasmid. The Plasmid group was treated with 1.5 μg/mL of the plasmid, while the nanoparticle-treated groups (PESPA@Plasmid and PESPA@CPPO@Plasmid) received nanoparticles at a concentration of 10 μg/mL. After 24 hours of treatment, the cells were observed and imaged using an inverted fluorescence microscope.

FIG. 13 presented the fluorescence microscopy images illustrating plasmid transfection efficiency in DC2.4 cells. The results demonstrated that the lipo2000 transfection reagent yielded minimal transfection efficacy in DC2.4 cells, with few GFP-positive cells observed. In contrast, both PESPA NPs and PESPA@CPPO NPs groups exhibited strong fluorescence signals, indicating that both nanoparticles significantly enhanced the plasmid transfection efficiency in DC2.4 cells.

Although the present disclosure has been described in detail above, it is not intended to be limited thereto. Those skilled in the art may make various modifications and changes without departing from the spirit and scope of the disclosure. Therefore, the protection scope of the present disclosure shall be defined by the appended claims.

Finally, it should be noted that as used herein, relational terms such as “first” and “second” are used solely to distinguish one entity or operation from another, rather than necessarily requiring or implying any actual relationship or sequence between such entities or operations. Furthermore, the term “comprising,” “including,” or any variation thereof is intended to cover a non-exclusive inclusion, such that a process, method, article or apparatus that includes a list of elements may include other elements not expressly listed or inherent to such process, method, article, or apparatus. Without further limitation, an element defined by the phrase “including a . . . ” does not exclude the presence of additional identical elements in the process, method, article, or apparatus that includes such element.

In this specification, various embodiments are described in a progressive manner, with the emphasis placed on illustrating the distinctions from other embodiments. For identical or similar parts shared among different embodiments, reference may be made to the corresponding descriptions in other embodiments.

The foregoing description of the disclosed embodiments is provided to enable those skilled in the art to make or use the present disclosure. Various modifications to these embodiments will be readily apparent to those skilled in the art, and the generic principles defined herein may be applied to other embodiments without departing from the spirit or scope of the disclosure. Therefore, the present disclosure is not intended to be limited to the embodiments described herein, but is to be accorded the broadest scope consistent with the principles and novel features disclosed herein.

What is claimed is:

1. A method for preparing a gene delivery nanoparticle, comprising:
subjecting 2-((tert-butoxycarbonyl)amino)ethyl methacrylate as a monomer, methoxy poly(ethyleneglycol)₅ₖ-reversible addition-fragmentation chain transfer agent (mPEG₅ₖ-RAFT) as a chain transfer agent and azobisisobutyronitrile as an initiator to a reversible addition-fragmentation chain-transfer polymerization reaction in dimethylformamide, followed by dialysis and freeze-drying to obtain an intermediate;
dissolving the intermediate in dichloromethane, followed by addition of trifluoroacetic acid and reaction to obtain polyethylene glycol-poly(aminoethyl methacrylate);
dissolving sparfloxacin in a hydrochloric acid solution, followed by addition of 1-(3-dimethylaminopropyl)-3-ethylcarbodiimide hydrochloride and N-hydroxysuccinimide to produce a first solution; dissolving the polyethylene glycol-poly(aminoethyl methacrylate) in dimethyl sulfoxide to produce a second solution; adding the second solution to the first solution to produce a mixture; and subjecting the mixture to dialysis and freeze-drying to obtain a final product polyethylene glycol-poly(sparfloxacin); and
placing the polyethylene glycol-poly(sparfloxacin) in an aqueous solution for self-assembly to form the gene delivery nanoparticle.

2. The method according to claim 1, wherein a concentration of the polyethylene glycol-poly(sparfloxacin) is 1-40 mg/mL.

3. The method according to claim 1, wherein the polyethylene glycol-poly(sparfloxacin) is capable of self-assembling into an empty nanoparticle or a bis[2,4,5-trichloro-6-(pentyloxycarbonyl)phenyl] oxalate (CPPO)-loaded nanoparticle in the presence of CPPO.

4. The method according to claim 3, wherein a concentration of CPPO is 0.5-20 mg/mL.

5. A gene delivery nanoparticle, wherein the gene delivery nanoparticle is prepared by the method according to claim 1.

6. The gene delivery nanoparticle according to claim 5, wherein a particle size of the gene delivery nanoparticle is 50-250 nm.

7. The gene delivery nanoparticle according to claim 5, wherein the gene delivery nanoparticle is capable of binding to immunoadjuvant cytosine-phosphate-guanine oligodeoxynucleotides (CpG ODN), programmed death ligand-1 small interfering RNA (PD-L1 siRNA) and ovalbumin (OVA) mRNA via electrostatic adsorption.

8. The gene delivery nanoparticle according to claim 7, wherein a weight ratio of the polyethylene glycol-poly(sparfloxacin) to the immunoadjuvant CpG ODN for binding is no less than 3:1, a weight ratio of the polyethylene glycol-poly(sparfloxacin) to the PD-L1 siRNA for binding is no less than 1:1, and a weight ratio of the polyethylene glycol-poly(sparfloxacin) to the OVA mRNA for binding is no less than 3:1.

9. The gene delivery nanoparticle according to claim 5, wherein the gene delivery nanoparticle is capable of serving as a carrier to transfect a plasmid into dendritic cells.
